(19) United States
(12) Patent Application Publication
(10) Pub. No.: US 2006/0142253 A1
(43) Pub. Date: Jun. 29, 2006

(54) Methods for treating neoplastic, angiogenic, vascular, fibroblastic, and/or immunosuppressive irregularities of the eye and/or joint via administration of combretastatin based medicaments, and iontophoretic devices for delivering combretastatin based medicaments

(76) Inventors: Thomas M. Parkinson, White Salmon, WA (US); Malgorzata Szlek, Salt Lake City, UT (US); Lindsay B. Lloyd, Salt Lake City, UT (US); David L. Vollmer, West Jordan, UT (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/359,925

(22) Filed: Feb. 21, 2006

Related U.S. Application Data
(62) Division of application No. 10/270,273, filed on Oct. 11, 2002, now abandoned.
(60) Provisional application No. 60/330,212, filed on Oct. 17, 2001.

(51) Int. Cl. A61K 31/66

(52) U.S. Cl. 514/130

(57) ABSTRACT

A method for treating neoplastic, angiogenic, vascular, fibroblastic, immunosuppressive, infectious, metabolic, and/or constitutional irregularities of the eye and/or joint of a living subject, comprising the steps of: providing a living subject, wherein the living subject includes an affected ocular and/or joint area having a neoplastic, angiogenic, fibroblastic, immunosuppressive, infectious, metabolic, and/or constitutional irregularity; providing a combretastatin-A4 based medicament, wherein the combretastatin-A4 based medicament is capable of preventing microtubule assembly, inhibiting the proliferation of endothelial, eukaryotic, and neoplastic cells, or altering their shape and function, as well as providing an anti-inflammatory effect; associating a therapeutically effective concentration of the combretastatin based medicament with the affected ocular and/or joint area of the living subject; and decreasing the neoplastic, angiogenic, vascular, fibroblastic, immunosuppressive, infectious, metabolic and/or constitutional irregularity of the eye and/or joint of the living subject.

### BACKGROUND OF THE INVENTION

1. Field of the Invention

The present invention relates in general to methods for treating neoplastic, angiogenic, vascular, fibroblastic, immunosuppressive, infectious, metabolic or constitutional irregularities of the eye and/or joint, and more particularly, to methods for treating the same via administration of combretastatin-A4 based medicament(s) which are capable of preventing microtubule assembly and thereby have the potential to inhibit eukaryotic, endothelial, and neoplastic cell proliferation, and may also provide anti-inflammatory effects. Beyond anti-proliferative and apoptotic effects on dividing vascular endothelial cells, CA4 has been shown to alter the shape of vascular endothelial cells thus diminishing blood flow in the microvasculature of solid tumors. The present invention further relates to the controlled administration of combretastatin-A4 based medicaments to an affected area of a living subject's eye and/or joint.

2. Background Art

Combretastatin based medicaments have been known in the art for 20 years, and several of these medicaments, particularly combretastatin-A4, have been shown to possess anti-proliferation and colchicine-like pharmacological action that may be exploited to provide anti-neoplastic, anti-angiogenic, antivascular, anti-fibroblastic and/or immunosuppressive activities, as well as providing anti-inflammatory effects. While administrating combretastatin-A4 based medicaments has been identified as being a promising remedy to treat many of the above-identified irregularities, delivering combretastatin-A4 based medicaments to an affected area of a patients eye or joint has remained heretofore largely problematic. Indeed, known prior art methods of administering combretastatin-A4 based medicaments, identified herein below, are replete with potential drawbacks and/or life threatening complications.

For example, delivering combretastatin-A4 based medicaments to an affected, local area of a living subjects eye using a systemic delivery method is problematic due to the potential for severe, sometimes life threatening, side effects associated with systemic delivery of combretastatin-A4 based medicaments, such as, for example, liver fibrosis, cirrhosis, leukopenia (bone marrow suppression), mucositis, ulcerative stomatitis, skin rash, nausea, abdominal distress, malaise, fatigue, chills and fever, diarrhea, gastrointestinal ulceration or perforation, pancreatitis, pericarditis, hypotension, deep venous thrombosis, thrombophlebitis, interstittial pneumonitis, headaches, drowsiness, cognitive dysfunction, reduced immunity, rash, photosensitivity, nephropathy, hematuria, alopecia, defective oogenesis, oligospermia, infertility, miscarriage, peripheral neurophathy, intra-ocular inflammation, and various forms of uveitis

Local delivery of combretastatin-A4 based medicaments via intraocular or intersynovial injection remains problematic for many reasons. Local intraocular injection is problematic in part because of the opportunity for, among other things, retinal detachment, bleeding into the interior of the eye, increased intraocular pressure, and increased risk of secondary infection. Although perhaps justifiable for occasional acute conditions, these risk factors render intraocular injection undesirable as a delivery mode for anything less than critically acute ocular irregularities. Furthermore, intraocular injections cannot only be scary and unpleasant, but also extremely painful for the patient. Similar drawbacks related to potential for bleeding, infection, and pain apply to repeated intrasynovial injections.

In addition to the above-identified problems, intraocular, peribulbar, and subconjuctival injection of combretastatin-A4 based medicaments can also be problematic, because such injections may not deliver sufficient quantities to the interior of the eye. Moreover, peribulbar, and subconjuctival, injections are demanding of the physician inasmuch as placement of the needle requires an extremely high level of precision.

Topical administration of combretastatin-A4 based medicaments to an affected, local area of a living subject's eye or joint is problematic due to its ineffectiveness for many applications, including affected areas in the back of the eye and interior area of the joint.

### SUMMARY OF THE INVENTION

The present invention is directed to a method for treating neoplastic, neovascular, vascular, fibroblastic, immunosuppressive, infectious, metabolic or constitutional irregularities of the eye and/or joint a living subject comprising the steps of: (a) providing a living subject, wherein the living subject includes an affected ocular and/or joint area having a neoplastic, angiogenic, vascular, fibroblastic, immunosuppressive, infectious, metabolic or constitutional irregularities of the eye and/or joint; (b) providing a combretastatin-A4 based medicament, wherein the combretastatin-A4 based medicament (particularly combretastatin-A4-3-O-phosphate, a combretastatin-A4 prodrug) capable of preventing microtubule assembly and inhibiting the proliferation of eukaryotic cells, including vascular, endothelial and neoplastic cells, or altering their shape and function, as well as providing anti-inflammatory effects; (c) associating a therapeutically effective concentration of the combretastatin-A4 based medicament with the affected ocular or joint area of the living subject via iontophoresis; and (d) decreasing the neoplastic, angiogenic, vascular, fibroblastic, immunosuppressive, infectious, metabolic or constitutional irregularities of the eye and/or joint of the living subject.

In a preferred embodiment of the present invention, the step of providing a combretastatin-A4 based medicament includes the step of providing a medicament represented by the following chemical structure: 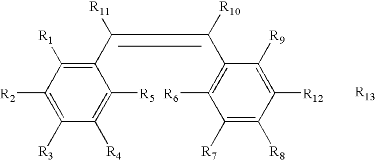
(Picture slightly distorted to maintain pictorial clarity) wherein R₁₋₁₁ are the same or different and comprise H, NH₂, a primary or secondary amino group, an amino acid, an azido group, a sulfonate group, a hydroxy group, an alkoxy group, a primary or secondary amino group, a straight or branched alkyl, cycloalkyl, polycycloalkyl, heterocycloalkyl, aryl, alkaryl, aralkyl, alkoxy, alkenyl, alkynyl group containing approximately 1 to approximately 25 carbon atom(s), a silyl or siloxyl group containing approximately 1 to approximately 25 silicon atom(s), and combinations thereof, and where R₁₁ and R₁₂ may be further joined through an additional bridging group to form a third ring constraining the molecule to the cis conformation, where the additional bridging group bond between R₁₁ and R₁₂ may contain S, N, or O, forming within the above structure for example, a thiophene ring, a pyrole ring, or a furan ring. Additionally, the entire bridge containing R₁₀ and R₁₁ between the two phenyl rings may be replaced by a ketone group to form a benzophenone structure. Alternatively, either phenyl ring may be exchanged in these structures for a napthyl or quinoline ring system with similar pendant groups as listed for the phenyl groups above. R₁₋₁₂ may comprise an ester of a pharmaceutically acceptable inorganic or organic polyprotic acid salt, amine salt, or amino acid salt such that the molecule above is rendered water soluble at or near physiologic pH. In a preferred embodiment, R₁₂ comprises PO₄³⁻, and R₁₃ comprises an atom or molecule comprising group I elements, group II elements, transition elements, or pharmaceutically acceptable organic counter ions, or any other combination of elements that together with the base molecule create a neutral molecule. In this embodiment, the combretastatin-A4 based medicament may comprise the structure:

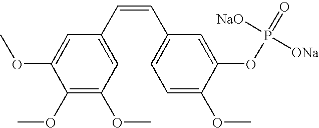

In another preferred embodiment of the present invention, the step of providing a combretastatin-A4 based medicament includes the step of providing (cis)-1-(3′,4′,5′,-trimethoxyphenyl)-2-(3″-hydroxy-4″-methoxyphenyl)ethene or the disodium salt of the 3″-phosphate ester pro drug of this embodiment.

In yet another preferred embodiment of the present invention, the step of associating a therapeutically effective concentration of the combretastatin-A4 based medicament with the living subject includes the step of ocular and/or transdermal iontophoretic delivery of the medicament in a concentration ranging from approximately 0.5 to 100 mg/ml in the electrode to achieve effective medicament tissue concentrations ranging from approximately 0.1 to approximately 1000 ng/ml per day as needed to prevent neovascularization or to treat abnormal vasculature of the tissue while avoiding potential toxic side-effects.

The present invention is also directed to a method for treating an affected area of a living subject's eye and/or joint, comprising the steps of: (a) associating a combretastatin-A4 based medicament with an ocular or transdermal iontophoretic device; (b) positioning at least a portion of the ocular or transdermal iontophoretic device on the eye or skin overlying the joint, respectively, of a living subject; and (c) iontophoretically delivering the combretastatin-A4 based medicament to an affected area of the living subject's eye or joint.

In a preferred embodiment of the present invention, the step of associating the combretastatin-A4 based medicament includes the step of associating a combretastatin-A4 based medicament capable of decreasing neoplastic, angiogenic, vascular, fibroblastic, immunosuppressive, infectious, metabolic and constitutional irregularities of the eye and joint of the living subject.

Preferably, the step of iontophoretically delivering the combretastatin-A4 based medicament includes delivering the same to at least one of the group consisting of either the sclera, ciliary body, iris, lens, cornea, aqueous fluid, vitreous body, retina, choroid, optic nerve, regions of the eye thereabout, or to stratum corneum, dermis, subcutaneous tissue, and joint synovia.

In accordance with the present invention, the step of iontophoretically delivering the combretastatin-A4 based medicament may include the step of iontophoretically delivering the combretastatin-A4 based medicament using a negative polarity current between approximately 0.1 mA and approximately 5 mA for a period of between approximately 1 and approximately 60 minutes.

The present invention is further directed to an ocular and/or transdermal iontophoretic device for delivering a combretastatin-A4 based medicament to an affected area of a living subject's eye and/or joint, comprising an active electrode assembly associated with a matrix, wherein the matrix includes a combretastatin-A4 based medicament capable of decreasing neoplastic, angiogenic, vascular, fibroblastic, immunosuppressive, infectious, metabolic, and/or constitutional irregularities of the eye and joint of the living subject.

In a preferred embodiment of the present invention, the ocular or transdermal iontophoretic device further comprises: (a) a counter electrode assembly, wherein the counter electrode assembly is configured for completing an electrical circuit between the active electrode assembly and an energy source; and (b) an energy source for generating an electrical potential difference.

In accordance with the present invention, the active ocular applicator electrode assembly may include a larger open-faced-low current density or smaller, high current density applicator electrode.

The present invention is also directed to an ocular or transdermal iontophoretic device for delivering a combretastatin-A4 based medicament to an affected area of a living subject's eye or joint, respectively, comprising: (a) a matrix, wherein the matrix is capable of temporarily retaining a solution having a combretastatin-A4 based medicament capable of decreasing neoplastic, angiogenic, vascular fibroblastic, immunosuppressive, infectious, metabolic, and/or constitutional irregularities of the eye of the living subject; (b) an active electrode assembly associated with the matrix, wherein the active electrode assembly is configured for iontophoretically delivering the combretastatin-A4 based medicament to the affected area of the living subject's eye or joint; (c) a counter electrode assembly, wherein the counter electrode assembly is configured for completing an electrical circuit between the active electrode assembly and an energy source; and (d) an energy source for generating an electrical potential difference.

The present invention further includes an ocular or transdermal iontophoretic device for delivering a combretastatin-A4 based medicament to an affected area of a living subject's eye or joint, comprising: (a) a reservoir, wherein the reservoir includes a combretastatin-A4 based medicament capable of decreasing neoplastic, angiogenic, vascular, fibroblastic, immunosuppressive, infectious, metabolic, and/or constitutional irregularities of the eye and joint of the living subject; (b) a matrix, wherein the matrix is capable of temporarily retaining a solution having a combretastatin-A4 based medicament; (c) an active electrode assembly associated with the matrix, wherein the active electrode assembly is configured for iontophoretically delivering the combretastatin-A4 based medicament to the affected area of the living subject's eye or joint; (d) a counter electrode assembly, wherein the counter electrode assembly is configured for completing an electrical circuit between the active electrode assembly and an energy source; and (e) an energy source for generating an electrical potential difference.

### BRIEF DESCRIPTION OF THE DRAWINGS

The invention will now be described with reference to the drawings wherein:

FIG. 1 of the drawings is a cross-sectional schematic representation of a first embodiment of an iontophoretic device fabricated in accordance with the present invention;

FIG. 2 of the drawings is a cross-sectional schematic representation of a first embodiment of an iontophoretic device fabricated in accordance with the present invention showing the association of a counter electrode assembly and an energy source; and

FIG. 3 of the drawings is a cross-sectional schematic representation of a second embodiment of an iontophoretic device fabricated in accordance with the present invention.

### DETAILED DESCRIPTION OF THE INVENTION

While this invention is susceptible of embodiment in many different forms, there is shown in the drawings and will herein be described in detail several specific embodiments with the understanding that the present disclosure is to be considered as an exemplification of the principles of the invention and is not intended to limit the invention to the embodiments illustrated.

It will be understood that like or analogous elements and/or components, referred to herein, may be identified throughout the drawings with like reference characters.

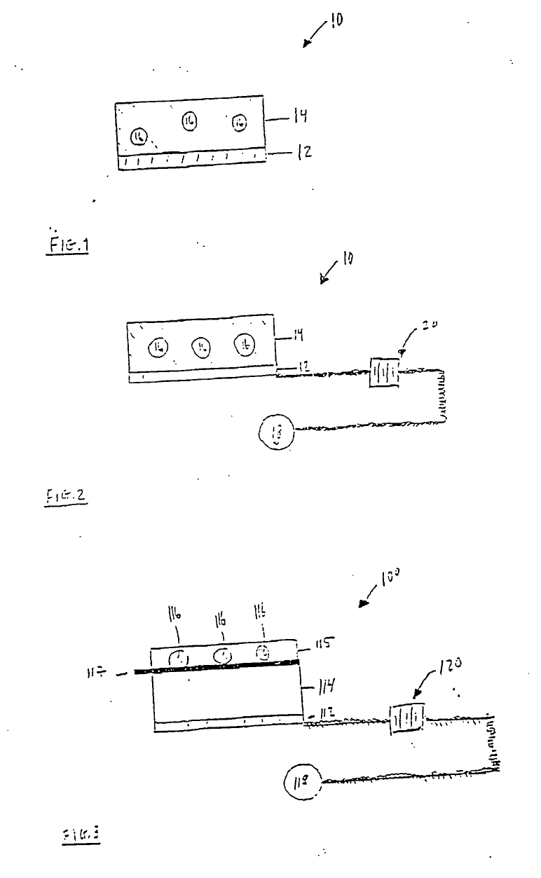

Referring now to the drawings and to FIG. 1 in particular, a first embodiment of iontophoretic device 10 is shown, which generally comprises active electrode assembly 12 and matrix 14. It will be understood that FIG. 1 is merely a cross-sectional schematic representation of iontophoretic device 10. As such, some of the components have been distorted from their actual scale for pictorial clarity. The iontophoretic device may be configured for application to either the eye or the skin overlying a joint of a living subject. As will be discussed in greater detail below, the iontophoretic device 10 is configured for delivering one or more combretastatin-A4 based medicament(s) which are capable of preventing microtubule assembly and either of inhibiting the proliferation of eukaryotic, endothelial and neoplastic cells, or altering their shape and function, therefore, treating, among other things, neoplastic, angiogenic, vascular, fibroblastic, immunosuppressive, infectious, metabolic, and/or constitutional irregularities of either the eye or joint of the living subject, or causing endothelial cells in the existing microvasculature to change shape (“round”) thereby blocking and causing atrophy of existing anomalous microvasculature. By iontophoretically administering a combretastatin-A4 based medicament to an affected area of a living subject's eye or joint, diseases and conditions associated with the above-identified ocular and joint irregularities can be efficiently remedied—especially including neovascular diseases of the eye wherein the affected area is toward the back of the eye, or generally proximate the optic nerve, and interior joint irregularities (of which there lacks a non-invasive method of treatment). Moreover, by utilizing iontophoretic technology, the living subject does not need to be exposed to such high medicament concentrations, which is of particular importance with such a potent classification of medicaments, because toxicity can occur rapidly using conventional, for example, systemic administration methods. Iontophoretic device 10 offers many advantages over the previously discussed prior art devices and associated delivery methods, including, but not limited to, simultaneous enabling of non-invasive and deep combretastatin-A4 based medicament delivery, non-invasive local delivery of an effective, therapeutic level of combretastatin-A4 based medicament while minimizing systemic concentrations, and enabling of, for example, sclera loading for prolonged ocular delivery (of controlled, sometimes low concentrations of medicaments) into regions in the back of the eye. Inasmuch as combretastatin-A4 is practically insoluble in aqueous solution and non-ionic, it is preferable that the combretastatin-A4 based medicaments be reacted through esterification with phosphate, producing a more soluble, and ionic compound, combretastatin-A4-3-O-phosphate. This compound has a water solubility of approximately 100 g/ml. The esterified compound, however, is no longer biologically active, and therefore requires an additional step for treatment of the named conditions.

Iontophoretic delivery of combretastatin-A4 based medicament to the eye of a living subject bypasses the blood-ocular barrier, providing therapeutically relevant levels of the medicament to intended regions while remaining non-invasive. The combretastatin-A4 based medicament first crosses the tear film and conjunctiva and then enters the sclera. If the esterified compound is used, as the combretastatin-A4-3-O-phosphate based medicament is distributed within the multiple inter-ocular compartments, and associated choroidal or retinal vasculature and endothelial cells where endogenous phosphatase enzymes convert the combrestastatin-A4-3-O-phosphate to the active form combretastatin-A4.

A similar process is conducted using a transdermal iontophoretic device, thus expanding the application of combretastatin-A4 based medicaments, and resulting benefits, to the joints of a living subject. For example, the process may result in the local treatment of rheumatoid inflammation in the joints of a living subject. The combretastatin-A4-3-O-phosphate based medicament is subcutaneously delivered via iontophoresis through the skin overlying the affected joint. The combretastatin-A4-3-O-phosphate is converted into active combretastatin-A4 by endogenous phosphatase enzymes within the subcutaneous tissue where it is substantially immobilized through loss of aqueous solubility in the local tissue later to be gradually taken up by the neovascular tissue in the synovia of the joint.

Active electrode assembly 12 generally comprises a conductive material, which upon application of an electrical potential difference thereto, drives an ionic combretastatin-A4 based medicament (i.e. an anionic or cationic medicament), received from matrix 14 and delivers the combretastatin-A4 based medicament into predetermined tissues and surrounding structures of a living subject's eye or joint. It will be understood that active electrode assembly 12 may comprise an anode or a cathode depending upon whether the medicament is cationic or anionic in form. (The preferred combretastatin-A4-3-O-phosphate is anionic at or above physiologic pH.) It will be further understood that active electrode assembly may include a low current density, open-faced or a smaller, high current density electrode. As would be readily understood to those having ordinary skill in the art, any one of a number of conventional active electrode assemblies is contemplated for use in accordance with the present invention. The only contemplated limitation relative to active electrode assembly 12 is that it must be geometrically and compositionally compatible for ocular or transdermal applications of living subjects, most relevantly, humans.

Matrix 14 extends contiguously from active electrode 12, and is preferably fabricated from a material capable of temporarily retaining combretastatin-A4 based medicament 16 in solution. The solution may also contain supplemental agents, such as electrolytes, stability additives, preserving additives, pH regulating buffers, etc. Matrix 14 may comprise, for example, a natural or synthetic amorphous member, a natural or synthetic sponge pad, a natural or synthetic lint free pad, a natural or synthetic low particulate member—just to name a few. Indeed, numerous other materials that would be known to those having ordinary skill in the art having the present disclosure before them are likewise contemplated for use, including monolithic or layered viscoelastic solid hydro gels or liquid reservoirs contained with microporous membranes. As with active electrode assembly 12, the only contemplated limitation relative to matrix 14 is that it must be geometrically and compositionally compatible for either ocular or transdermal applications of living beings, most relevantly, humans.

An aqueous, mixed aqueous, or polar solution of medicament 16 is retained within matrix 14. In accordance with the present invention, ionic medicament 16 comprises one or more combretastatin-A4 based medicament(s) that are capable of treating, among other things, neoplastic, angiogenic, vascular, fibroblastic, immunosuppressive, infectious, metabolic, and constitutional irregularities of the eye and joint.

Such combretastatin-A4 based medicaments may be represented by the following chemical structure: 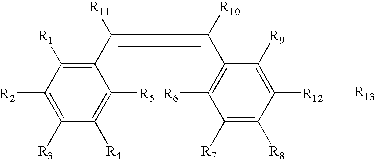
(picture distorted slightly for pictorial clarity) wherein R₁₋₁₁ are the same or different and comprise H, NH₂, a primary or secondary amino group, an azido group, a sulfonate group, a hydroxy group, an alkoxy group, a primary or secondary amino group, a straight or branched alkyl, cycloalkyl, polycycloalkyl, heterocycloalkyl, aryl, alkaryl, aralkyl, alkoxy, alkenyl, alkynyl group containing approximately 1 to approximately 25 carbon atom(s), a silyl or siloxyl group containing approximately 1 to approximately 25 silicon atom(s), and combinations thereof, and where R₁₁ and R₁₂ may be further joined through an additional bridging group to form a third ring constraining the molecule to the cis conformation, where the additional bridging group bond between R₁₁ and R₁₂ may contain S, N, or O, forming within the above structure for example a thiophene ring, a pyrole ring, or a furan ring. Additionally, the entire bridge containing R₁₀ and R₁₁ between the two phenyl rings may be replaced by a ketone group to form a benzophenone structure. Alternatively, either phenyl ring may be exchange for a napthyl or quinoline ring system with similar pendant groups as listed for the phenyl groups above. R₁₋₁₂ may comprise an ester of a pharmaceutically acceptable inorganic or organic polyprotic acid salt or an amine salt such that the molecule above is rendered water soluble at or near physiologic pH. In a preferred embodiment, R₁₂ comprises PO₄³⁻, and R₁₃ comprises an atom or molecule comprising group I elements, group II elements, transition elements, or pharmaceutically acceptable organic counter ions, or any other combination of elements that together with the base molecule create a neutral molecule.

For example, the combretastatin-A4 based medicament may comprise the structure:

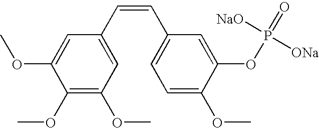

In a preferred embodiment of the present invention, the combretastatin-A4 based medicaments may include (cis)-1-(3,4,5,-trimethoxyphenyl)-2-(3-hydroxy-4-methoxyphenyl)ethene, derivatives and analogs thereof.

As is shown in FIG. 2, iontophoretic device 10 may also include counter electrode assembly 18 and energy source 20. Counter electrode assembly 18 may be housed within iontophoretic device 10, or alternatively, may be remotely associated with iontophoretic device 10 via conventional electrical conduit. Counter electrode assembly 18 is configured for completing an electrical circuit between active electrode assembly 12 and energy source 20. As with active electrode 12, counter electrode 18 may comprise an anode or a cathode depending upon whether the medicament is cationic or anionic in form. As would be readily understood to those having ordinary skill in the art, any one of a number of counter electrodes is contemplated for use in accordance with the present invention.

Similarly counter electrode assembly 18 and energy source 20 may be housed within iontophoretic device 10, or alternatively, may be remotely associated with iontophoretic device 10 via conventional electrical conduit. Energy source 20 for generating an electrical potential difference preferably supplies low voltage constant direct current between approximately 0.1 milliamps (mA) and approximately 5 mA. The energy source may also provide for an initial higher voltage during current ramp-up to break down higher initial tissue resistance as in commercial power supply units used for transdermal iontophoresis. For purposes of the present disclosure, energy source 20 may include one or more primary or secondary electrochemical cells. While specific examples of energy source 20 have been disclosed, for illustrative purposes only, it will be understood that other energy sources known to those having ordinary skill in the art having the present disclosure before them are likewise contemplated for use.

Referring now to the drawings and to FIG. 3 in particular, a second embodiment of an iontophoretic device 100 is shown, which generally comprises active electrode assembly 112, matrix 114, reservoir 115, counter electrode assembly 118, and energy source 120. The iontophoretic device 100 may be configured for application to either the eye or joint. It will be understood that active electrode assembly 112, matrix 114, counter electrode assembly 118, and energy source 120, are configured analogously to previously discussed active electrode assembly 12, matrix 14, counter electrode assembly 18, and energy source 20, respectively. Iontophoretic device 100 is configured for delivering a combretastatin-A4 based medicament to an affected area of a living subject's eye or joint for treating neoplastic, angiogenic, vascular, fibroblastic, and/or immunosuppressive irregularities of the eye or joint.

Reservoir 115 includes combretastatin-A4 based medicament 116, in solution, which is capable of treating the above-identified ocular or joint irregularities. Reservoir 115 may include a releasable cover member 117 that, upon articulation, releases combretastatin-A4 based medicament 116 into matrix 114. Such a release cover enables prompt delivery of the combretastatin-A4 based medicament with very little device preparation.

The present invention is also directed to a method for treating an affected area of a living subject's eye and/or joint comprising the following steps. First, a combretastatin-A4 based medicament is associated with an ocular iontophoretic device. Preferably, the combretastatin-A4 based medicament is metered from a syringe or single unit dose. Second, at least a portion of the ocular iontophoretic device is positioned on the eye or skin overlying the joint of a living being. Finally, the combretastatin-A4 based medicament is iontophoretically delivered to an affected area of the living subject's eye or joint. Preferably, the delivery lasts between approximately 1 and approximately 60 minutes to the eye and/or 1 minute to 12 hours to the skin over the affected joint. Compared to prior art administration or delivery methods, the present invention enables a generally painless, non-invasive, and deep delivery of the combretastatin-A4 based medicament. Moreover, the combretastatin-A4 based medicament is locally delivered to an affected area of a living subject's eye or joint at an effective, therapeutic level. Preferred ocular delivery regions include the sclera, ciliary body, iris, lens, cornea, aqueous fluid, vitreous body, retina, choroids, optic nerve, and regions of the eye thereabout. Preferred transdermal delivery regions include stratum corneum, dermis, subcutaneous tissue, and joint synovia.

For purposes of the present disclosure, neoplastic, angiogenic, vascular, fibroblastic, immunosuppressive, infectious, metabolic, and constitutional irregularities of the eye of a living subject can also be treated in accordance with the following method. First, a living subject with a neoplastic, angiogenic, vascular, fibroblastic, immunosuppressive, infectious, metabolic, and constitutional irregularity is provided. Second, one or more of the above-identified combretastatin-A4 based medicaments is provided. Third, a therapeutically effective concentration of the combretastatin-A4 based medicament is associated with and/or administered to the affected ocular or joint area of the living subject. Preferably, the combretastatin-A4 based medicament is administered in a concentration ranging from approximately 0.5 to approximately 50 mg/ml. The duration of a single application may range from 1 minute to approximately 60 minutes for ocular application and from 15 minutes to 12 hours for transdermal delivery to an affected joint. The medicament may be administered on a schedule ranging from once every day to once every 30 days. The duration of combretastatin-A4 based therapy may range from a single application to multiple applications that are administered over a period of months to years, depending upon the disease being treated. Upon administration of the combretastatin based medicament, the neoplastic, angiogenic, vascular, fibroblastic, immunosuppressive, infectious, metabolic, and constitutional irregularity of the eye and/or joint of the living subject is materially decreased.

It will be understood that while iontophoresis has been disclosed as one suitable means for the local ocular and/or joint administration of combretastatin-A4 based medicaments, any one of a number of other local administering means are likewise contemplated for use, such as via needle injection and/or topical administration from a suitable delivery vehicle or applicator device formulated to promote passive diffusion of the active substance.

For ocular applications, combretastatin-A4-3-O-phosphate is dissolved in aqueous solution, for example, in a concentration near 5% w/v, or if less, with a suitable non-ionic excipient (such as polyethylene glycol or glycerin) to retain a preferred osmolality of approximately 300±30 mOsm/kg. The solution may be buffered with other salts, such as phosphate, carbonate, or citrate. The pH is adjusted to a value similar to physiologic and also to where the medicament is fully ionized, preferably between 6.8 and 8, and most preferably pH 7.4, using buffer solution, NaOH or HCl. The final concentration of combretastatin is between 0.5 and 50 mg/ml. Iontophoretic current is applied at 1.0 to 4.0 milliamperes for 1 to 60 minutes. It will be understood to those having ordinary skill in the art that the previously identified formulation, although being preferred, is not the only formulation that can be used.

For transdermal applications to an affected joint, combretastatin-A4-3-O-phosphate is dissolved in aqueous solution, for example, in a concentration of from 0.1 to 100 mg/ml. The solution may be buffered with other pharmaceutically acceptable salts, such as phosphate, carbonate, or citrate. The pH is adjusted to where the medicament is fully ionized, preferably between pH 7.4 and 9, using buffer solution, NaOH or HCl. Iontophoretic current is applied at 1.0 to 4.0 milliamperes for 1 minute to 12 hours. It will be understood to those having ordinary skill in the art that the previously identified formulation, though being preferred, is not the only formation that can be used.

The foregoing description merely explains and illustrates the invention and the invention is not limited thereto except insofar as the appended claims are so limited, as those skilled in the art who have the disclosure before them will be able to make modifications without departing the scope of the invention.

1-24. (canceled)

25. A method for achieving an effect in a living subject, comprising: administering an effective amount of a combretastatin-A4 based medicament to the living subject via iontophoresis, wherein the effect is decreasing a neoplastic, angiogenic, fibroblastic, immunosuppressive, infectious, metabolic, and/or constitutional ocular or joint irregularity of the living subject.

26-30. (canceled)

31. The method according to claim 25, wherein the combretastatin-A4 based medicament is represented by the following chemical structure: 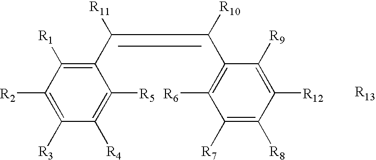
wherein R₁₋₁₁ are the same or different and comprise H, NH₂, a primary or secondary amino group, an azido group, a sulfonate group, a hydroxy group, an alkoxy group, a primary or secondary amino group, a straight or branched alkyl, cycloalkyl, polycycloalkyl, heterocycloalkyl, aryl, alkaryl, aralkyl, alkoxy, alkenyl, alkynyl group containing approximately 1 to approximately 25 carbon atom(s), a silyl or siloxyl group containing approximately 1 to approximately 25 silicon atom(s), and combinations thereof, R₁₂ comprises an ester of a pharmaceutically acceptable inorganic or organic polyprotic acid salt or an amine salt such that the molecule above is rendered water soluble at or near physiologic pH, and R₁₃ comprises an atom or molecule comprising group I elements, group II elements, transition elements, or pharmaceutically acceptable organic counter ions, or any other combination of elements that together with the base molecule create a neutral molecule.

32. The method according to claim 25, wherein the combretastatin-A4 based medicament is represented by the following chemical structure:

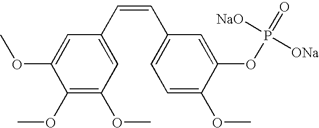

33. The method according to claim 25, wherein the combretastatin-A4 based medicament is (cis)-1-(3,4,5,-trimethoxyphenyl)-2-(3-hydroxy-4-methoxyphenyl)ethene and derivatives thereof.

34. The method according to claim 25, wherein the administration of the effective amount of combretastatin-A4 based medicament to the living subject is at a concentration ranging from approximately 0.5 to approximately 100 mg/ml per day for approximately 1 to approximately 30 days.
